(19) United States
(12) Patent Application Publication
(10) Pub. No.: US 2024/0210411 A1
(43) Pub. Date: Jun. 27, 2024

(54) COMPOUNDS COMPRISING AN ISOBARIC REGION, AN ENRICHMENT HANDLE, AND A THIOL-REACTIVE GROUP, AND METHODS OF USING THE SAME

(71) Applicant: IQ Proteomics LLC, Framingham, MA (US)

(72) Inventors: Craig Braun, Newton, MA (US); Brian K. Erickson, Brookline, MA (US); Ryan Kunz, Framingham, MA (US)

(73) Assignee: IQ Proteomics LLC, Framingham, MA (US)

(21) Appl. No.: 18/285,674

(22) PCT Filed: Apr. 8, 2022
(86) PCT No.: PCT/US2022/024035

§ 371 Date: Oct. 5, 2023

Related U.S. Application Data
(60) Provisional application No. 63/172,572, filed on Apr. 8, 2021.

(51) Int. Cl. G01N 33/68; C07K 17/06; G01N 33/543

(52) U.S. Cl. CPC G01N 33/6845; C07K 17/06; G01N 33/54306; G01N 33/54386; G01N 33/6848; G01N 2333/976; G01N 2440/12; G01N 2458/15

(57) ABSTRACT

Compounds comprising an isobaric region, an enrichment handle, and a thiol-reactive group are generally described. Methods of using the same (e.g., performing multiplexed quantitative analysis of the reactivity of one or more cysteine residues across multiple samples simultaneously) are also described.

### RELATED APPLICATIONS

This application claims priority under 35 U.S.C. § 119(e) to U.S. Provisional Patent Application No. 63/172,572, filed Apr. 8, 2021, which is hereby incorporated herein by reference in its entirety.

### TECHNICAL FIELD

Compounds comprising an isobaric region, an enrichment handle, and a thiol-reactive group, and methods of using the same, are generally described.

### SUMMARY

Compounds comprising an isobaric region, an enrichment handle, and a thiol-reactive group are generally described. Methods of using the same (e.g., performing multiplexed quantitative analysis of the reactivity of one or more cysteine residues across multiple samples simultaneously) are also described. The subject matter of the present disclosure involves, in some cases, interrelated products, alternative solutions to a particular problem, and/or a plurality of different uses of one or more systems and/or articles.

Some aspects relate to compounds. In some embodiments, the compound comprises moiety X (X); moiety Y (Y); and moiety Z (Z); wherein moiety X comprises an isobaric reagent, moiety Y comprises an enrichment handle; and moiety Z comprises a thiol reactive group. Some aspects relate to collections of compounds. In some embodiments, in the collection of compounds, each compound comprises moiety X (X); moiety Y (Y); and moiety Z (Z); wherein moiety X comprises an isobaric region, moiety Y comprises an enrichment handle; and moiety Z comprises a thiol-reactive group; wherein moiety Y is the same in each compound in the collection, wherein moiety Z is the same in each compound in the collection, and wherein moiety X differs only in a different placement of heavy isotopes for at least two compounds in the collection.

In certain embodiments, each compound further comprises one or more linkers. According to some embodiments, the one or more linkers comprises an aliphatic chain. In accordance with certain embodiments, the aliphatic chain comprises an alkane or heteroalkane chain. In some embodiments, the aliphatic chain comprises the heteroalkane chain, and the heteroalkane chain comprises a polyethylene glycol chain.

In some embodiments, Y comprises a hexapeptide of histidine residues, a phosphorylated region, a fluoroalkane,

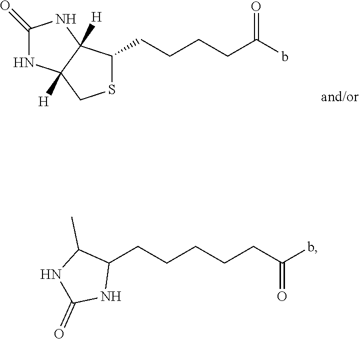

where “b” indicates the point of attachment to the remainder of the compound. For example, in certain cases, Y comprises

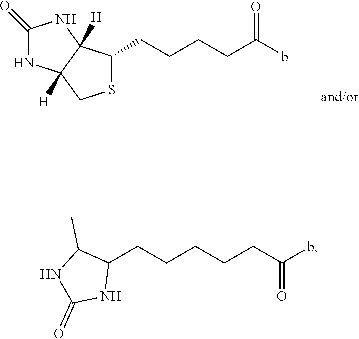

where “b” indicates the point of attachment to the remainder of the compound.

According to certain embodiments, Z comprises iodoacetamide, maleimide, chloroacetamide, and/or acrylamide.

In accordance with some embodiments, X comprises a moiety of the formula:

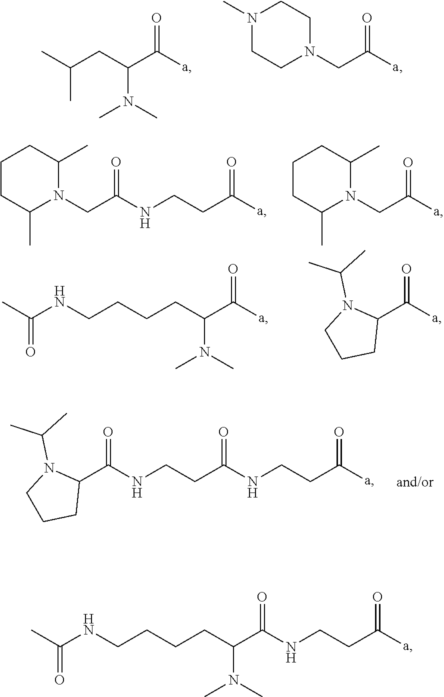

or isotopically enriched derivatives thereof, where “a” represents the point of attachment to the remainder of the compound.

In certain embodiments, the compounds are of the formula:

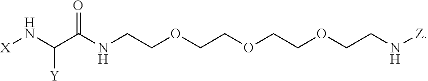

In some embodiments, the compounds are of the formula:

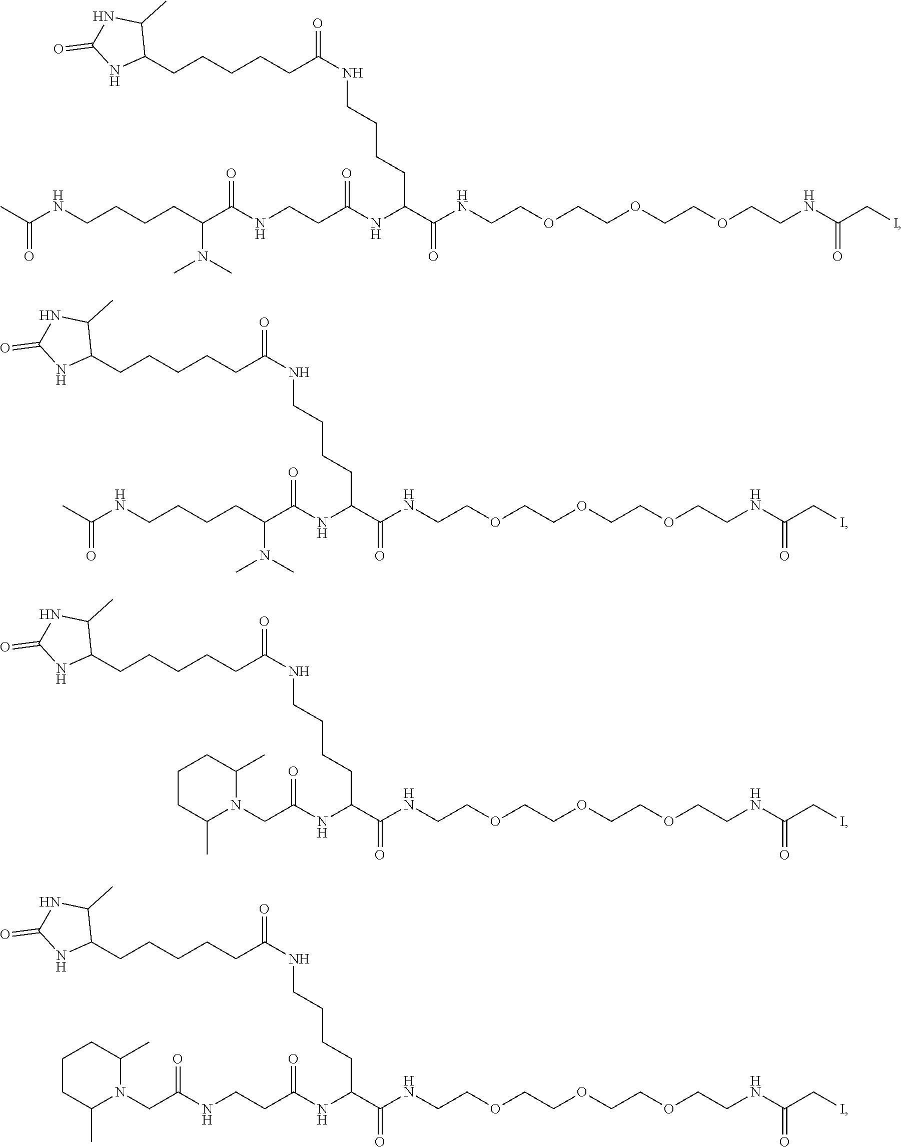 [SMILES: CC(=O)NCCCCC(C(=O)NCCC(=O)NC(CCCCNC(=O)CCCCCC1NC(=O)NC1C)C(=O)NCCOCCOCCOCCNC(=O)CI)N(C)C ; CC(=O)NCCCCC(C(=O)NC(CCCCNC(=O)CCCCCC1NC(=O)NC1C)C(=O)NCCOCCOCCOCCNC(=O)CI)N(C)C ; CC1NC(=O)NC1CCCCCC(=O)NCCCCC(NC(=O)CN1C(C)CCCC1C)C(=O)NCCOCCOCCOCCNC(=O)CI ; CC1NC(=O)NC1CCCCCC(=O)NCCCCC(NC(=O)CCNC(=O)CN1C(C)CCCC1C)C(=O)NCCOCCOCCOCCNC(=O)CI]

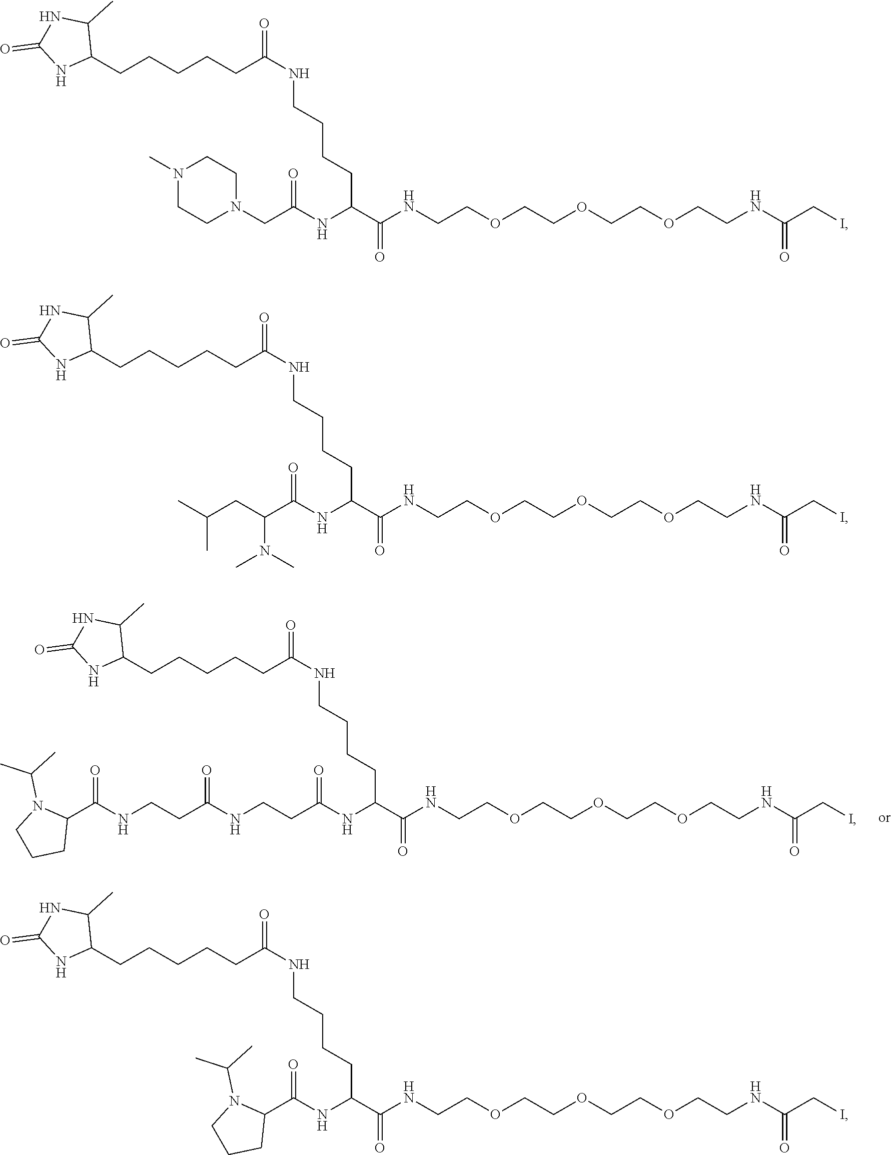 [SMILES: CC1NC(=O)NC1CCCCCC(=O)NCCCCC(NC(=O)CN1CCN(C)CC1)C(=O)NCCOCCOCCOCCNC(=O)CI ; CC(C)CC(C(=O)NC(CCCCNC(=O)CCCCCC1NC(=O)NC1C)C(=O)NCCOCCOCCOCCNC(=O)CI)N(C)C ; CC1NC(=O)NC1CCCCCC(=O)NCCCCC(NC(=O)CCNC(=O)CCNC(=O)C1CCCN1C(C)C)C(=O)NCCOCCOCCOCCNC(=O)CI ; "or" ; CC1NC(=O)NC1CCCCCC(=O)NCCCCC(NC(=O)C1CCCN1C(C)C)C(=O)NCCOCCOCCOCCNC(=O)CI]

or isotopically enriched derivatives thereof.

Some aspects relate to systems. In certain embodiments, the system comprises a collection of compounds and a substrate to which moiety Y selectively binds.

Some aspects relate to methods of using the collection of compounds. In some embodiments, the method comprises combining a first compound of the collection of compounds with a first sample comprising a first protein and/or peptide in a first container, and combining a second compound of the collection of compounds with a second sample comprising a second protein and/or peptide in a second container.

In some embodiments, the method comprises combining a first compound of the collection of compounds with a first sample comprising a first protein and/or peptide in a first container, and combining a second compound of the collection of compounds with a second sample comprising a second protein and/or peptide in a second container; wherein each compound in the collection of compounds comprises moiety X (X); moiety Y (Y); and moiety Z (Z); wherein moiety X comprises an isobaric region, moiety Y comprises an enrichment handle; and moiety Z comprises a thiol-reactive group; wherein moiety Y is the same in each compound in the collection, wherein moiety Z is the same in each compound in the collection, and wherein moiety X differs only in a different placement of heavy isotopes for at least two compounds in the collection.

In certain embodiments, the first compound of the collection of compounds covalently attaches to one or more cysteine residues of the first protein and/or peptide and/or the second compound of the collection of compounds covalently attaches to one or more cysteine residues of the second protein and/or peptide.

According to some embodiments, the method further comprises combining the contents of the first container and the second container into a third container. In certain cases, the method further comprises reducing and/or alkylating at least one cysteine residue on the proteins and/or peptides. In some instances, the method further comprises digesting at least one of the proteins and/or peptides. In accordance with some embodiments, the reduction, alkylation, and digestion steps occur after the combination of the samples with the compounds of the collection. In certain embodiments, the reduction, alkylation, and digestion steps occur after the combining of the contents of the first container and the second container into the third container.

In accordance with some embodiments, the method further comprises contacting the contents of the third container with a substrate, such that moiety Y preferentially binds to the substrate. In certain cases, the substrate comprises avidin, streptavidin, a chelated metal, and/or a fluorous resin. In some instances, the contacting the contents of the third container with the substrate occurs after the reduction, alkylation, digestion, and combining the samples with the compounds of the collection steps.

According to certain embodiments, the method further comprises analyzing the samples with mass spectrometry. In some cases, the analyzing the samples with mass spectrometry occurs after the reduction, alkylation, digestion, contacting the contents of the containers with the immobilized substrate, and combining the samples with the compounds of the collection steps.

In some embodiments, the method comprises analyzing multiple samples simultaneously. For example, in certain cases, the method comprises analyzing greater than or equal to 5 samples simultaneously. In some instances, the method comprises analyzing less than or equal to 100 samples simultaneously.

In accordance with certain embodiments, the method further comprises determining whether a target is bound to one or more cysteine residues on the one or more proteins and/or peptides. According to some embodiments, the target is a pharmaceutical drug candidate and/or the method comprises screening a library of pharmaceutical drug candidates.

Some aspects relate to kits comprising a collection of compounds. In some embodiments, the kit comprises instructions for use. In certain cases, the kit further comprises one or more buffers. In some instances, the kit further comprises one or more microcentrifuge tubes. In accordance with certain embodiments, the kit further comprises one or more spin filters. In some embodiments, the kit further comprises a control sample. In certain embodiments, the kit further comprises a substrate to which moiety Y (Y) selectively binds.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a compound, in accordance with certain embodiments, covalently attached to a cysteine residue of a peptide and/or protein via a thiol-reactive group, and selectively bound to a substrate via an enrichment handle.

FIG. 2A shows isobaric reagents used in the comparative example of Example 1.

FIG. 2B shows 6 trifunctional compounds, in accordance with certain embodiments, as used in Example 1.

FIG. 2C shows the comparative workflow used in Example 1.

FIG. 2D shows a novel workflow, in accordance with certain embodiments, used in Example 1.

FIG. 2E shows the results of the comparative workflow (lower panel) compared to a novel workflow, in accordance with certain embodiments (upper panel).

FIG. 3 shows a workflow (left), in accordance with certain embodiments, and a comparative workflow (right).

### DETAILED DESCRIPTION

Compounds comprising an isobaric region, an enrichment handle, and a thiol-reactive group are generally described. Methods of using the same (e.g., performing multiplexed quantitative analysis of the reactivity of one or more cysteine residues across multiple samples simultaneously) are also described.

In some embodiments, the compounds disclosed herein may be used as tags for protein and/or peptide samples. The proteins and/or peptides may, for example, comprise at least 5 amino acids, at least 6 amino acids, at least 7 amino acids, at least 10 amino acids, at least 20 amino acids, at least 50 amino acids, or at least 100 amino acids. In certain instances where multiple samples are analyzed, each sample may be combined with a compound within a collection of compounds. For example, in some cases, each sample may be combined with a compound within a collection of compounds through covalent attachment of a cysteine residue of the protein and/or peptide sample by a thiol-reactive group on the compound. In certain embodiments, the compounds within the collection of compounds are identical except for an isobaric region. In certain instances, the isobaric region of each compound is structurally identical to the isobaric regions of the other compounds but is isotopically distinct. In some embodiments, each isobaric region has a reporter region having a unique molecular weight. In some cases, the reporter region facilitates determination of the origin of a particular peptide residue. In accordance with certain embodiments, each isobaric region has a balancing region having a unique molecular weight. According to some embodiments, the reporter region and balancing region are selected such that the overall molecular weights of the isobaric regions are identical. In some cases, having identical overall molecular weights of the isobaric regions ensures that the tagged peptides and/or proteins remain together after mass-based separation.

In accordance with some embodiments, the methods disclosed herein comprise tagging each protein and/or peptide sample (e.g., with a compound disclosed herein). In certain embodiments, the method comprises combining all of the tagged protein and/or peptide samples (e.g., in one container), digesting them (e.g., with a protease), and separating (e.g., using chromatographic separation, such as liquid chromatography) each peptide type into a separate fraction. According to some embodiments, once the peptides have been fractionated, the method comprises breaking the bonds between the tags and the peptides. In some embodiments, the method comprises using mass spectrometry (e.g., for each peptide fraction) to determine the relative abundance of each tag, which informs upon the relative abundance of that peptide within each proteome. In certain embodiments, the method comprises performing multiplexed analysis as numerous samples can be analyzed simultaneously.

In certain embodiments, the methods disclosed herein have one or more advantages (e.g., due to the tri-functionality of the compound), including reduced number of steps (e.g., a single step for attachment, enrichment, and quantification of cysteine residues), reduced time for sample preparation, simplified workflow (e.g., no need to label free amines of digested peptides with mass tagging reagents and/or samples can be mixed prior to protease digestion), reduced experimental variability, increased reproducibility, increased throughput (e.g., more samples can be analyzed simultaneously), and/or increased yield (e.g., from increased labeling and/or reduced side reactions).

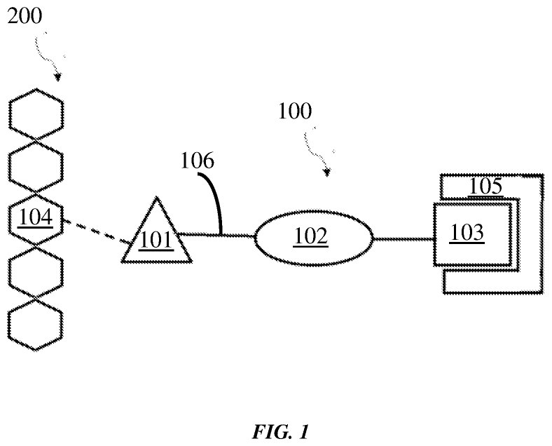

Some aspects relate to compounds (e.g., a collection of compounds). In some embodiments, the compound (e.g., each compound in the collection of compounds) comprises moiety X (X), moiety Y (Y), and moiety Z (Z). According to certain embodiments, the compound comprises one or more linkers. For example, in FIG. 1, in accordance with some embodiments, compound 100 comprises moiety Z 101, moiety X 102, moiety Y 103, and linker 106. It should be understood that FIG. 1 is schematic only, and that the 101, 102, and 103 regions may be arranged in other ways. For example, in some embodiments, regions 101, 102, and 103 are all connected to a central hub.

A compound comprising moiety X (X), moiety Y (Y), and moiety Z (Z) may also be referred to as a trifunctional compound. As used herein, a trifunctional compound comprises at least three moieties but may have more, in some embodiments. For example, in some cases, a trifunctional compound comprises at least three or more moieties each having a distinct function.

In certain embodiments, moiety X (X) comprises an isobaric region. For example, in FIG. 1, in accordance with some embodiments, moiety X 102 comprises an isobaric region. According to some embodiments, moiety X differs only in a different placement of heavy isotopes for at least two compounds (e.g., at least three, at least five, at least seven, or all) in the collection. In some embodiments, the isobaric regions are differentially isotopically labeled such that one or more (e.g., two or more, three or more, five or more, seven or more, or all) of the isobaric regions are isobaric and chromatographically indistinguishable, but yield signature or reporter ions (e.g., with mass spectrometry) that can be used to identify and quantify individual isobaric regions. In some instances, the presence of the isobaric region facilitates quantification (e.g., multiplexed quantification).

In certain embodiments, the isobaric region comprises a reporter region and/or a balancing region. In some embodiments, each reporter region (e.g., in two or more, three or more, five or more, seven or more, or all of a collection of compounds) has a unique molecular weight. In accordance with certain embodiments, each balancing region (e.g., in two or more, three or more, five or more, seven or more, or all of a collection of compounds) has a unique molecular weight. According to some embodiments, the reporter region and balancing region are selected such that the overall molecular weights of the isobaric regions are identical (e.g., to ensure that the tagged peptides and/or proteins remain together after mass-based separation).

In some embodiments, moiety X (X) and/or the isobaric region comprises a moiety of the formula:

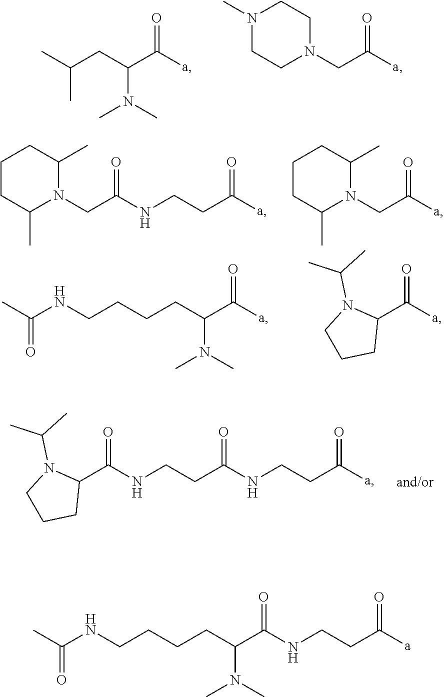

where “a” represents the point of attachment to the remainder of the compound. For example, in certain cases, a represents the point of attachment to one of the one or more linkers. In some embodiments, moiety X (X) and/or the isobaric region comprises an isotopically enriched derivative of the moieties shown above. For example, in some cases, moiety X (X) and/or the isobaric region comprises a moiety identical to those shown above but with isotopic enrichment at one or more positions. Other isobaric regions are also possible, such as those disclosed in International Patent Application Publication No. WO 2016/196,994, filed Jun. 3, 2016, which is hereby incorporated herein by reference in its entirety.

According to some embodiments, moiety Y (Y) comprises an enrichment handle. For example, in FIG. 1, in accordance with some embodiments, moiety Y 103 comprises an enrichment handle. In certain embodiments, moiety Y is the same in each compound in the collection of compounds. In some embodiments, the enrichment handle comprises a moiety that has affinity for a substrate such that the moiety selectively binds to the substrate. For example, in some cases, the enrichment handle hydrogen bonds to the substrate and/or interacts with the substrate through other non-covalent interactions, van der Waals interactions (such as fluorous interactions and van der Waals interactions other than fluorous interactions), and/or electrostatic interactions. In certain embodiments, the enrichment handle comprises a moiety that has affinity for a substrate such that the moiety selectively binds to the substrate in such a way that facilitates separation of peptides bound to the substrate from peptides that are not. For example, in FIG. 1, in accordance with some embodiments, moiety Y 103 is selectively bound to substrate 105 (e.g., through a non-covalent interaction).

In some embodiments, the enrichment handle comprises a moiety that has affinity (e.g., a selective binding affinity) for a substrate that is capable of being immobilized on a solid support. Examples of a solid support include a resin, a wall of a well plate such as a 96/384 well plate, and/or a chromatographic column bed. For example, in certain embodiments, moiety Y (Y) and/or the enrichment handle comprises a biotin-like and/or desthiobiotin-like moiety. An example of a biotin-like and/or desthiobiotin-like moiety is a moiety that would result from a reaction involving biotin and/or desthiobiotin. Biotin and/or desthiobiotin has affinity for avidin and/or streptavidin, which is capable of being immobilized on a solid support (e.g., resin), in some cases.

According to some embodiments, the enrichment handle preferentially binds the substrate. For example, in accordance with some embodiments, the equilibrium dissociation constant (K_{D}) between the enrichment handle and the substrate is greater than or equal to 1×10⁻²⁰ mol/L, greater than or equal to 1×10⁻¹⁸ mol/L, greater than or equal to 1×10⁻¹⁶ mol/L, greater than or equal to 1×10⁻¹⁵ mol/L, greater than or equal to 1×10⁻¹³ mol/L, or greater than or equal to 1×10⁻¹⁰ mol/L. In certain cases, the equilibrium dissociation constant (K_{D}) between the enrichment handle and the substrate is less than or equal to 1×10⁻⁵ mol/L, less than or equal to 1×10⁻⁶ mol/L, less than or equal to 5×10⁻⁷ mol/L, less than or equal to 1×10⁻⁷ mol/L, less than or equal to 1×10⁻⁸ mol/L, less than or equal to 1×10⁻⁹ mol/L, or less than or equal to 1×10⁻¹⁰ mol/L. Combinations of these ranges are also possible (e.g., greater than or equal to 1×10⁻²⁰ mol/L and less than or equal to 1×10⁻⁵ mol/L, or greater than or equal to 1×10⁻¹⁶ mol/L and less than or equal to 5×10⁻⁷ mol/L). The dissociation constant may be determined according to isothermal calorimetry at atmospheric pressure, 25° C., and 7.4 pH in aqueous solutions.

In certain embodiments, the enrichment handle has specific affinity for the substrate. In some embodiments, the substrate can comprise a support material and a selective binding agent to which the enrichment handle selectively binds. In some cases, the enrichment handle has at least 5-fold (or at least 10-fold, at least 20-fold, or at least 100-fold, and/or, in some embodiments, up to 10¹²-fold, or more) higher affinity for the substrate when the selective binding agent is present in the substrate, relative to the enrichment handle's affinity for the substrate when the selective binding agent is not present but the substrate is otherwise identical.

As another example of the enrichment handle's specific affinity for the substrate, in certain instances, the enrichment handle has at least 5-fold (or at least 10-fold, at least 20-fold, or at least 100-fold, and/or, in some embodiments, up to 10¹²-fold, or more) higher affinity for the substrate than the affinity of at least one protein (or all proteins) in the sample for the substrate. As a further example, in some instances, the enrichment handle has at least 5-fold (or at least 10-fold, at least 20-fold, or at least 100-fold, and/or, in some embodiments, up to 10¹²-fold, or more) higher affinity for the substrate than the affinity of at least one buffer (or all buffers) in the sample for the substrate. In some instances, the enrichment handle has at least 5-fold (or at least 10-fold, at least 20-fold, or at least 100-fold, and/or, in some embodiments, up to 1012-fold, or more) higher affinity for the substrate than the affinity of all other compounds in the sample for the substrate.

In some embodiments, the enrichment handle has selective affinity for the substrate. For example, in certain instances, an entity containing the enrichment handle has at least 5-fold (or at least 10-fold, at least 20-fold, or at least 100-fold, and/or, in some embodiments, up to 10¹²-fold, or more) higher affinity for the substrate compared to the affinity the same entity without the enrichment handle has for the substrate. As an example, in some cases, one or more protein and/or peptide being analyzed has at least 5-fold (e.g., at least 10-fold, at least 20-fold, or at least 100-fold, and/or, in some embodiments, up to 10¹²-fold, or more) higher affinity for the substrate when covalently attached to a compound comprising the enrichment handle than the affinity the same protein and/or peptide without the compound comprising the enrichment handle has for the substrate. As another example, in some cases, a trifunctional compound (e.g., any of the trifunctional compounds described herein) can have at least 5-fold (e.g., at least 10-fold, at least 20-fold, or at least 100-fold, and/or, in some embodiments, up to 10¹²-fold, or more) higher affinity for the substrate than the affinity the same compound without the enrichment handle would have for the substrate.

According to some embodiments, the enrichment handle can be released from the substrate with reasonable yield. For example, in some cases, the enrichment handle is released from the substrate with a yield of at least 70%, at least 80%, at least 90%, at least 95%, or at least 98%. In accordance with certain embodiments, the presence of moiety Y (Y) and/or the enrichment handle facilitates separation of peptides covalently attached to a compound disclosed herein from peptides that are not covalently attached. As an example, in some cases, the presence of moiety Y (Y) and/or the enrichment handle facilitates separation of peptides covalently attached to a compound disclosed herein from peptides that are not covalently attached on a resin to which the affinity substrate is immobilized. In certain embodiments, once the peptides covalently attached to a trifunctional compound disclosed herein are separated from the peptides that are not, the peptides covalently attached to the compound are released (e.g., with reasonable yield) from the substrate.

Examples of enrichment handles include a hexapeptide of histidine residues (“His tag”) (which has affinity to chelated metal resins, such as nickel and/or cobalt resins), a phosphorylated region such as a HPO4 modification (which has affinity to chelated metal resins, such as titanium and/or iron resins), a fluoroalkane (which has affinity to fluorous resins), and/or a biotin-like and/or desthiobiotin-like moiety (which has affinity to avidin and/or streptavidin), such as

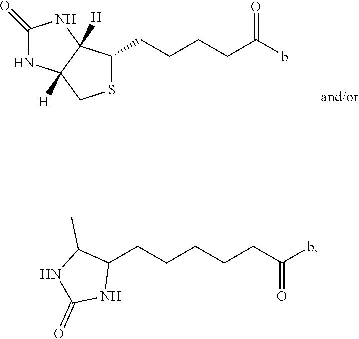

where “b” indicates the point of attachment to the remainder of the compound. In certain embodiments, the enrichment handle has a molecular weight of less than 1500 Da, less than or equal to 1250 Da, less than or equal to 1000 Da, less than or equal to 750 Da, or less than or equal to 500 Da. In some embodiments, the enrichment handle has a molecular weight of greater than or equal to 100 Da, greater than or equal to 150 Da, or greater than or equal to 200 Da. Combinations of these ranges are also possible (e.g., greater than or equal to 100 Da and less than 1500 Da, greater than or equal to 100 Da and less than or equal to 1000 Da, or greater than or equal to 150 Da and less than or equal to 500 Da).

In accordance with some embodiments, moiety Z (Z) comprises a thiol-reactive group. For example, in FIG. 1, in accordance with some embodiments, moiety Z 101 comprises a thiol-reactive group. As used herein, a thiol-reactive group is a group that reacts with a thiol. In some embodiments, moiety Z (Z) is a group that covalently attaches to a thiol group, such as a thiol group on a cysteine. For example, in FIG. 1, in accordance with some embodiments, moiety Z 101 is covalently attached to the thiol of cysteine residue 104 on protein and/or peptide 200. In certain embodiments, moiety Z (Z) and/or the thiol-reactive group comprises iodoacetamide, maleimide, chloroacetamide, and/or acrylamide. In some instances, moiety Z is the same in each compound in the collection of compounds.

In some embodiments, the one or more linkers comprises an aliphatic chain. For example, in FIG. 1, in accordance with some embodiments, linker 106 comprises an aliphatic chain. Examples of aliphatic chains include alkane chains and/or heteroalkane chains. Examples of heteroalkane chains include polyethylene glycol chains. In some instances, one or more of the one or more linkers is cleavable. In certain embodiments, one or more of the one or more linkers is chemically labile. In some cases, one or more of the one or more linkers separates two or more of moiety X (X), moiety Y (Y), and/or moiety Z (Z).

In accordance with certain embodiments, the compound is (and/or the compounds of the collection are each) of the formula:

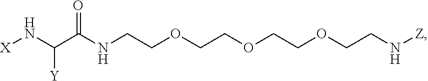

where X, Y, and Z are as described elsewhere herein for moiety X, moiety Y, and moiety Z, respectively.

In some embodiments, the compound and/or a moiety of the compound—such as moiety X, moiety Y, and/or moiety Z—is isotopically enriched. For example, in some cases, moiety X is isotopically enriched. In certain cases, the compound and/or a moiety of the compound—such as moiety X, moiety Y, and/or moiety Z—is isotopically enriched with a stable isotope. For example, in certain instances, moiety X is isotopically enriched with a stable isotope.

According to some embodiments, the compound is (and/or the compounds of the collection are each) of the formula:

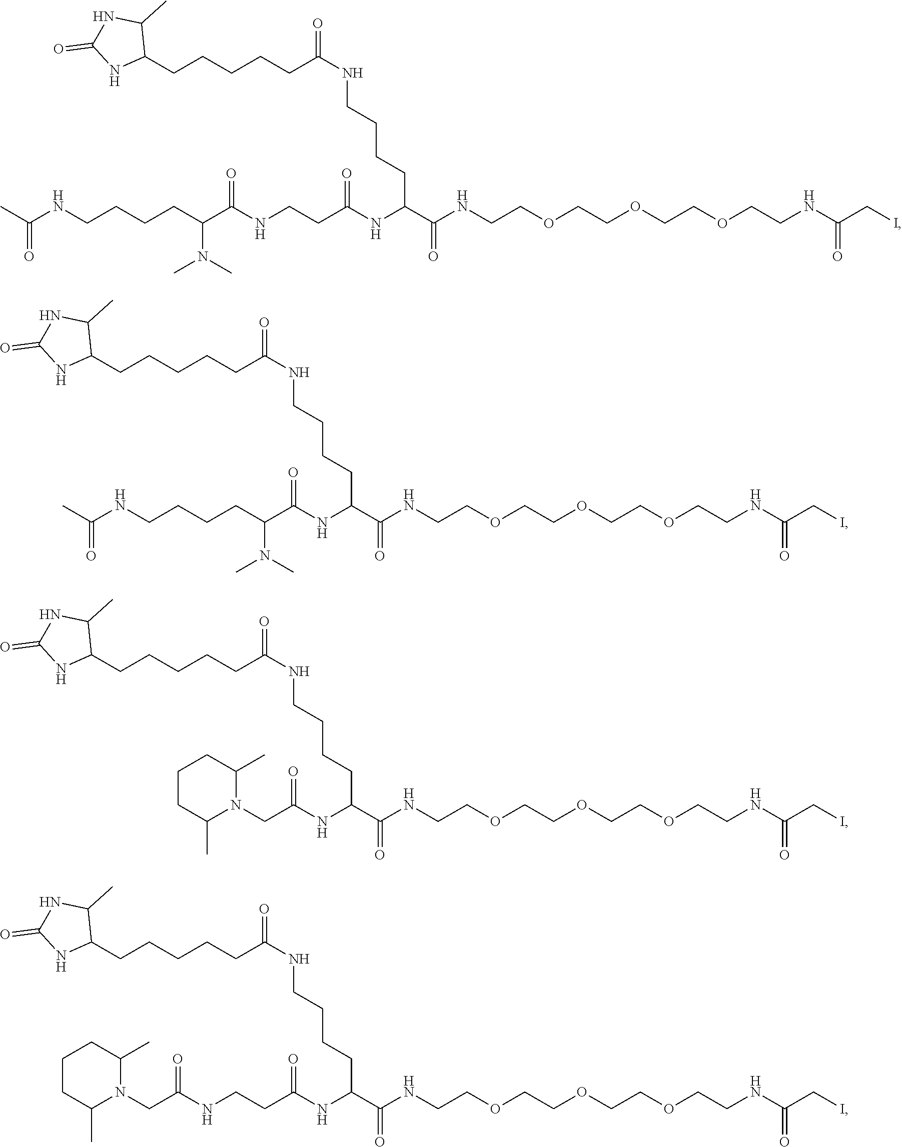 [SMILES: CC(=O)NCCCCC(C(=O)NCCC(=O)NC(CCCCNC(=O)CCCCCC1NC(=O)NC1C)C(=O)NCCOCCOCCOCCNC(=O)CI)N(C)C ; CC(=O)NCCCCC(C(=O)NC(CCCCNC(=O)CCCCCC1NC(=O)NC1C)C(=O)NCCOCCOCCOCCNC(=O)CI)N(C)C ; CC1NC(=O)NC1CCCCCC(=O)NCCCCC(NC(=O)CN1C(C)CCCC1C)C(=O)NCCOCCOCCOCCNC(=O)CI ; CC1NC(=O)NC1CCCCCC(=O)NCCCCC(NC(=O)CCNC(=O)CN1C(C)CCCC1C)C(=O)NCCOCCOCCOCCNC(=O)CI]

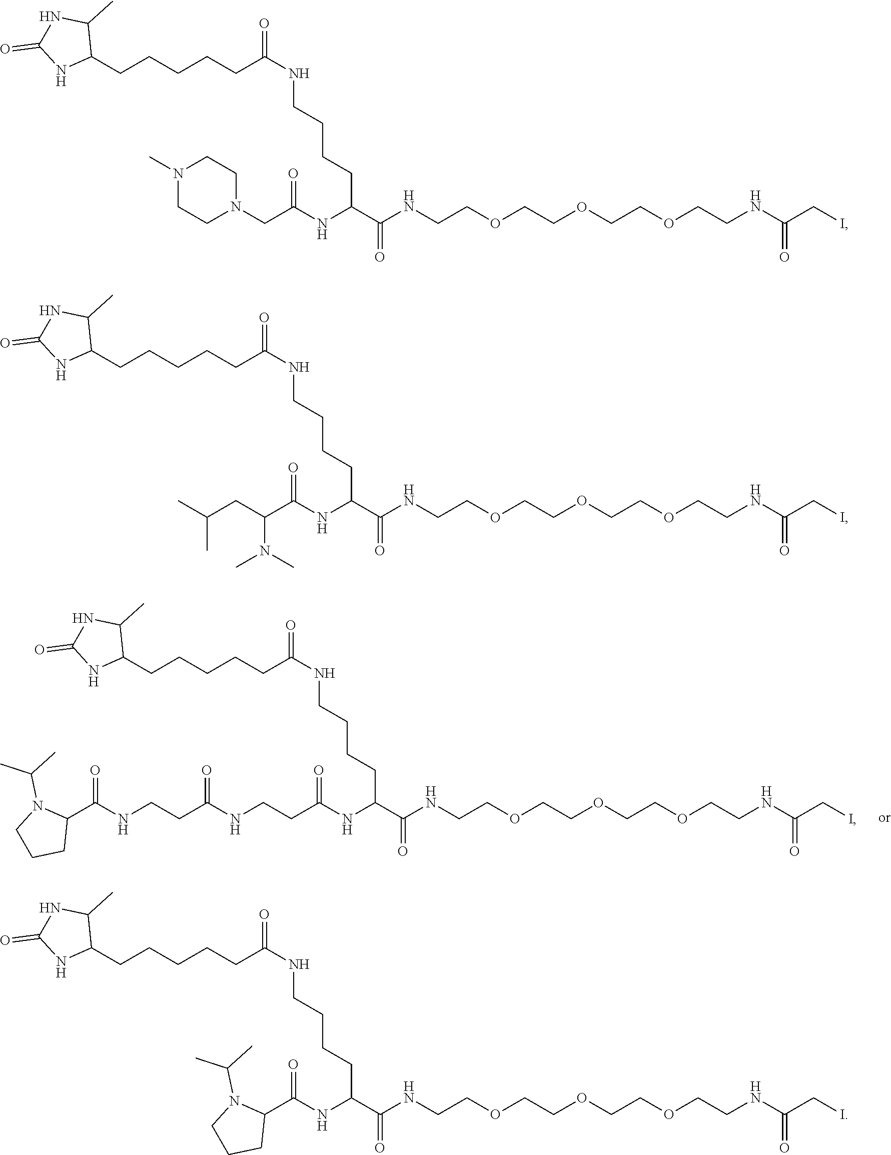 [SMILES: CC1NC(=O)NC1CCCCCC(=O)NCCCCC(NC(=O)CN1CCN(C)CC1)C(=O)NCCOCCOCCOCCNC(=O)CI ; CC(C)CC(C(=O)NC(CCCCNC(=O)CCCCCC1NC(=O)NC1C)C(=O)NCCOCCOCCOCCNC(=O)CI)N(C)C ; CC1NC(=O)NC1CCCCCC(=O)NCCCCC(NC(=O)CCNC(=O)CCNC(=O)C1CCCN1C(C)C)C(=O)NCCOCCOCCOCCNC(=O)CI ; "or" ; CC1NC(=O)NC1CCCCCC(=O)NCCCCC(NC(=O)C1CCCN1C(C)C)C(=O)NCCOCCOCCOCCNC(=O)CI]

In some embodiments, the compound is an isotopically enriched derivative of the formulas shown above. For example, in certain cases, the compound is identical to those shown above but with isotopic enrichment at one or more positions.

In certain embodiments, the compound has a molecular weight of less than or equal to 1500 Da, less than or equal to 1250 Da, less than or equal to 1000 Da, less than or equal to 750 Da, or less than or equal to 500 Da. In some embodiments, the compound has a molecular weight of greater than or equal to 400 Da, greater than or equal to 500 Da, or greater than or equal to 600 Da. Combinations of these ranges are also possible (e.g., greater than or equal to 400 Da and less than or equal to 1500 Da, or greater than or equal to 400 Da and less than or equal to 1000 Da).

Some aspects are methods. In some embodiments, the method comprises using the compounds disclosed herein.

According to some embodiments, the method comprises combining one or more compounds of the collection of compounds with one or more samples comprising a protein and/or peptide. For example, in certain cases, the method comprises combining one or more compounds of the collection of compounds with one or more samples comprising a protein and/or peptide in such a way that the compound covalently attaches to one or more cysteine residues on the protein and/or peptide. In certain embodiments, the method comprises attaching (e.g., covalently attaching) the compound (e.g., moiety Z (Z) and/or the thiol-reactive group) to one or more thiol groups. According to some embodiments, the method comprises attaching (e.g., covalently attaching) the compound (e.g., moiety Z (Z) and/or the thiol-reactive group) to one or more cysteine residues (e.g., of one or more proteins and/or peptides). For example, in some cases, the method comprises covalently attaching moiety Z and the thiol-reactive group of the compound to one or more thiol groups on one or more cysteine residues on one or more proteins and/or peptides.

For example, in some cases, the method comprises combining a first compound of the collection of compounds with a first sample comprising a first protein and/or peptide in a first container, and combining a second compound of the collection of compounds with a second sample comprising a second protein and/or peptide in a second container. In certain embodiments, the first compound of the collection of compounds covalently attaches to one or more cysteine residues of the first protein and/or peptide (e.g., via moiety Z (Z) and the thiol-reactive group of the first compound) and/or the second compound of the collection of compounds covalently attaches to one or more cysteine residues of the second protein and/or peptide (e.g., via moiety Z (Z) and/or the thiol-reactive group of the second compound).

According to some embodiments, the method further comprises combining the contents of the first container and the second container into a third container. In some instances, the method further comprises reducing and/or alkylating at least one cysteine residue on the proteins and/or peptides. In certain cases, the method further comprises digesting at least one of the proteins and/or peptides. For example, in some instances, the method comprises protease digesting, such as trypsin digesting, at least one of the proteins and/or peptides to form peptides. In accordance with some embodiments, the reduction, alkylation, and/or digestion steps occur after the combination of the samples with the compounds of the collection, such as after covalently attaching a cysteine residue of the protein and/or peptide to moicty Z (Z) and/or a thiol-reactive group. According to certain embodiments, the reduction, alkylation, and/or digestion steps occur after the combining of the contents of the first container and the second container into the third container.

In some embodiments, the method comprises isolating cysteine-containing peptides (e.g., in mixtures). In certain cases, the isolating cysteine-containing peptides occurs after digestion of the proteins. In certain embodiments, the method comprises isolating cysteine-containing peptides using chromatographic separation, such as liquid chromatography. In accordance with some embodiments, the method comprises isolating cysteine-containing peptides using moiety Y (Y) and the enrichment handle. For example, in certain cases, a substrate (e.g., avidin and/or streptavidin) for which the enrichment handle (e.g., biotin and/or desthiobiotin) has affinity (e.g., selective binding affinity) is immobilized on a solid support (e.g., a chromatography resin), and the method comprises adding the peptides (e.g., after digestion of proteins) to the solid support (e.g., a chromatography resin). For example, in some instances, a substrate for which the enrichment handle has affinity is immobilized on a chromatography resin, and the method comprises adding the peptides to the chromatography resin, such that the peptides attached to the compounds disclosed herein bind or otherwise adhere to the chromatography resin while the peptides that are not attached to the compounds disclosed herein pass through.

For example, in some embodiments, the method comprises contacting the contents of the third container with a substrate, such that moiety Y preferentially binds to the substrate. As discussed in more details above, examples of suitable substrate may include avidin, streptavidin, a chelated metal, and/or a fluorous resin. In certain embodiments, contacting the contents of the third container with the substrate occurs after the reduction, alkylation, digestion, and/or combining the samples with the compounds of the collection steps. For example, in some embodiments, contacting the contents of the third container with the substrate occurs after the reduction, alkylation, digestion, and combining the samples with the compounds of the collection steps.

According to certain embodiments, the method comprises quantifying the abundance (e.g., relative and/or absolute) of cysteine-containing peptides. For example, in certain instances, the method comprises quantifying the abundance of cysteine-containing peptides after isolating the cysteine-containing peptides. In some cases, the method comprises quantifying the abundance (e.g., relative and/or absolute) of cysteine-containing peptides across multiple samples (e.g., in high throughput). In certain instances, the method comprises quantifying the abundance of cysteine-containing peptides using mass spectrometry (e.g., LC-MS).

According to some embodiments, the method comprises analyzing the samples with mass spectrometry. In certain cases, the analyzing the samples with mass spectrometry occurs after the reduction, alkylation, digestion, contacting the contents of the containers with the immobilized substrate, and/or combining the samples with the compounds of the collection steps. For example, in some instances, the analyzing the samples with mass spectrometry occurs after the reduction, alkylation, digestion, contacting the contents of the containers with the immobilized substrate, and combining the samples with the compounds of the collection steps.

In some embodiments, the method comprises determining whether a target is bound to one or more cysteine residues on one or more proteins and/or peptides. For example, in certain embodiments, the proteins and/or peptides are combined with a target prior to performing the methods disclosed herein. In some such embodiments, the compounds disclosed herein bind to free cysteine residues but do not bind to cysteine residues that are bound to the target. In some such embodiments, the methods disclosed herein are performed to determine which cysteine residues are bound to the compounds disclosed herein, which allows determination of which cysteine residues are bound to the target.

An example of a suitable target is a pharmaceutical drug candidate. In some embodiments, the pharmaceutical drug candidate is a compound under investigation for treating or preventing a disease or condition in a subject, such as a human and/or other subject disclosed herein. In some instances, the pharmaceutical drug candidate is approved for treating or preventing a disease or condition in a subject but is under investigation for treating or preventing a different disease or condition in a subject. In some embodiments, the pharmaceutical drug candidate is capable of covalently binding to cysteine. In certain embodiments, the pharmaceutical drug candidate comprises an electrophile. In certain cases, the pharmaceutical drug candidate comprises an organic molecule and/or a peptide. For example, in some instances, the pharmaceutical drug candidate comprises an organic molecule having a molecular weight of less than or equal to 1500 g/mol (e.g., less than or equal to 1250 g/mol, less than or equal to 1000 g/mol, less than or equal to 750 g/mol, or less than or equal to 500 g/mol, and/or, in some embodiments, as little as 100 g/mol, 50 g/mol, 30 g/mol, or less). In certain embodiments, the pharmaceutical drug candidate comprises a peptide having a molecular weight of less than or equal to 5000 g/mol (e.g., less than or equal to 4000 g/mol, less than or equal to 3000 g/mol, or less than or equal to 2000 g/mol, and/or, in some embodiments, as little as 100 g/mol, 50 g/mol, 30 g/mol, or less). In some cases, the method comprises screening a library of targets, such as pharmaceutical drug candidates.

In accordance with certain embodiments, the method comprises performing multiplexed quantitative analysis. For example, in some cases, the method comprises performing multiplexed quantitative analysis of the reactivity of the one or more cysteine residues (e.g., across multiple samples). In certain instances, the method comprises analyzing (e.g., performing multiplexed quantitative analysis of the reactivity of the one or more cysteine residues across multiple samples) multiple samples simultaneously.

For example, in some embodiments, the multiple samples and/or the mixtures described herein comprise greater than or equal to 1, greater than or equal to 3, greater than or equal to 5, greater than or equal to 10, greater than or equal to 16, greater than or equal to 20, greater than or equal to 24, greater than or equal to 32, greater than or equal to 40, or greater than or equal to 48 samples. In certain embodiments, the multiple samples and/or the mixtures described herein comprise less than or equal to 100, less than or equal to 75, less than or equal to 50, less than or equal to 48, less than or equal to 40, less than or equal to 32, less than or equal to 24, less than or equal to 20, or less than or equal to 16 samples. Combinations of these ranges are also possible (e.g., greater than or equal to 1 and less than or equal to 100, greater than or equal to 5 and less than or equal to 50, or greater than or equal to 16 and less than or equal to 48).

In some embodiments, the method comprises isotope enrichment (e.g., with stable isotopes). For example, in certain embodiments, the method comprises isotope enrichment of the compound and/or a moiety thereof, such as moiety X, moiety Y. and/or moiety Z.

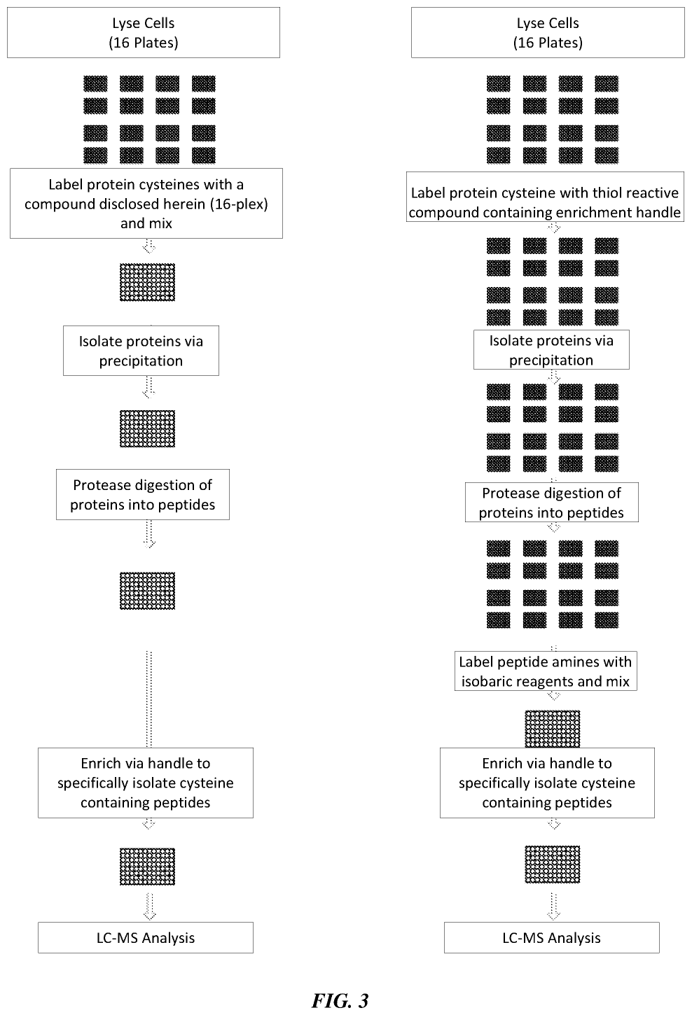

FIG. 3 shows an example of a method disclosed herein (left) and a comparator method (right), each processing 1536 whole cell lysates (arrayed into 16 96-well plates) and characterizing cysteine peptides by LC-MS. As shown in FIG. 3, in some embodiments, the methods disclosed herein have advantages over comparator methods, such as the ability to mix (multiplex) samples much earlier in sample preparation workflows, which may, in some cases, increase throughput by reducing the number of sample manipulations required to process a given number of samples and/or eliminate some sources of variability in sample processing between samples by ensuring that all samples in a given mixture are subjected to the exact same conditions during sample preparation once mixed.

Certain embodiments relate to kits. In some cases, a kit comprises a compound and/or collection of compounds (e.g., any compound and/or collection of compounds disclosed herein). In accordance with some embodiments, the kit comprises instructions for use. For example, in some cases, the kit comprises instructions for any method disclosed herein. In certain instances, the kit comprises one or more buffers. In some embodiments, the kit comprises one or more microcentrifuge tubes. According to certain embodiments, the kit comprises one or more spin filters. In certain instances, the kit comprises substrate that selectively binds with the enrichment handle and/or moiety Y (Y) of one or more (e.g., all) of the compounds in the kit. In some cases, the equilibrium dissociation constant between the enrichment handle (or the compounds comprising the enrichment handle) and the substate is lower than the equilibrium dissociation constant between the substrate and any other compound (and/or any other component) of the kit. In accordance with certain embodiments, the kit comprises one or more control samples. In some embodiments, the control sample comprises a protein and/or peptide comprising a cysteine residue. In certain cases, the instructions for use provide a method for using the control sample to verify that a compound and/or collection of compounds provided in the kit are not defective in one or more ways.

The term “isotopes” refers to variants of a particular chemical element such that, while all isotopes of a given element share the same number of protons in each atom of the element, those isotopes differ in the number of neutrons.

The terms “treatment,” “treat,” and “treating” refer to reversing, alleviating, delaying the onset of, or inhibiting the progress of a disease. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent.” “preventing.” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

A “subject” (e.g., to which administration is contemplated) refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal.

Examples of diseases include but are not limited to a genetic disease, proliferative disease (e.g., cancer), inflammatory disease, autoimmune disease, disease of an organ (e.g., liver, spleen, lung, heart, and/or kidney), hematological disease, neurological disease, psychiatric disorder, metabolic disorder, immune disorder, CNS disorder, a bacterial disease, a viral disease, a parasitic disease, and/or a fungal disease.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH2F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

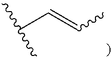

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C8), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and lor 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃-14 carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (Cs), cyclopentenyl (Cs), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1 ]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C10), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur. Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1.4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The following examples are intended to illustrate certain embodiments of the present invention, but do not exemplify the full scope of the invention.

### EXAMPLE 1

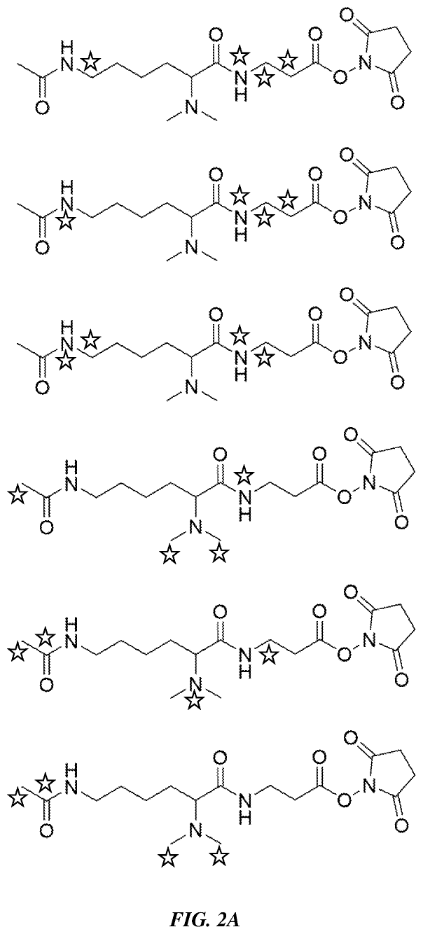

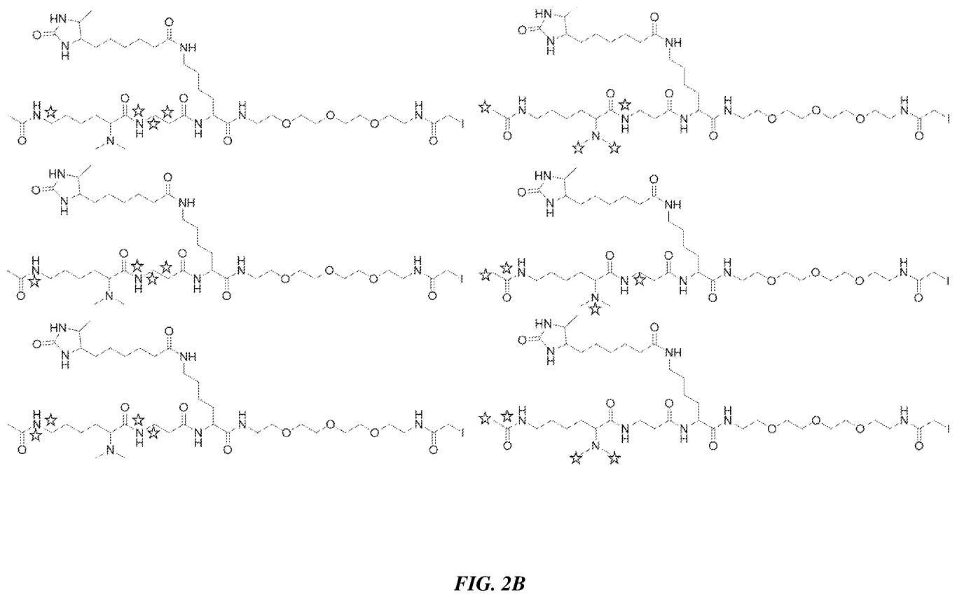

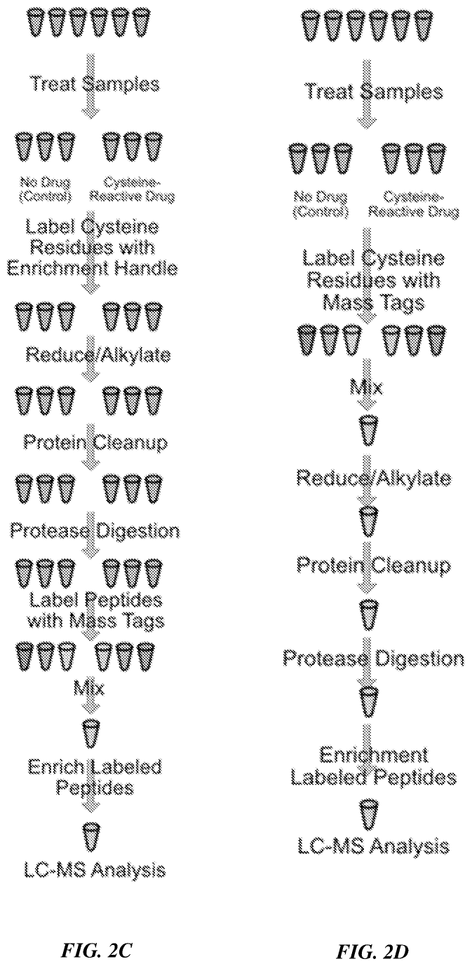

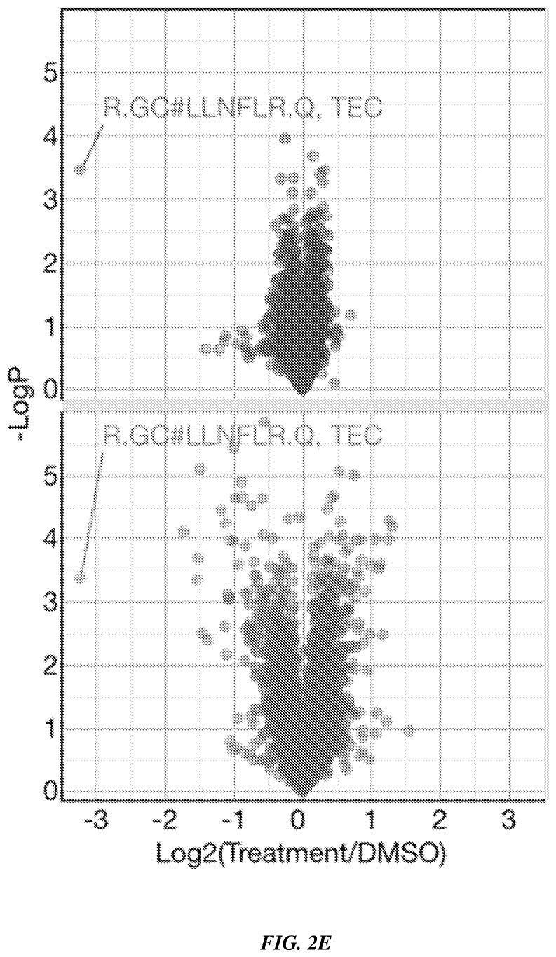

A set of 6 isotopically distinguishable tags, as shown in FIG. 2B, were synthesized. The stars in FIG. 2B show the positions that have been isotopically enriched. The performance of these tags for identification of the target of a cysteine-directed covalent therapeutic (Ibrutinib) was compared to the results obtained using a comparative cysteine enrichment workflow using isobaric regions (labeling peptide amines, FIG. 2A) for multiplexed quantification in combination with desthiobiotin polyethylene oxide iodoacetamide for labeling of cysteine residues and enrichment of cysteine peptides.

In the comparative workflow (FIG. 2C), six aliquots of a whole cell lysate were treated in sets of three with either Ibrutinib (luM) or vehicle (DMSO) for labeling of reactive cysteine residues in proximity to the Ibrutinib binding site of proteins with which it interacts. All remaining reactive cysteine residues were then labeled with desthiobiotin iodoacetamide polyethylene oxide. Inaccessible (disulfide bound) cysteine residues were reduced and alkylated with DTT/Iodoacetamide, and proteins were digested with trypsin. Digested peptides were labeled with isobaric regions, mixed, and the mixture was incubated with streptavidin agarose for enrichment of cysteine-containing peptides. Samples were analyzed by LC-MS, and the relative abundance of each cysteine-containing peptide across the mixed samples was quantified using the isobaric region reporter ions. Protein targets of Ibrutinib were identified by a reduction in signal in the samples treated with the drug, corresponding to drug engagement at the cysteine of interest, and concomitant reduction in the amount of signal for the desthiobiotin labeled peptide (FIG. 2E (lower panel), #—desthiobiotin modification).

In the novel workflow disclosed herein (FIG. 2D), six aliquots of a whole cell lysate were treated in sets of three with either Ibrutinib (1 uM) or vehicle (DMSO) for labeling of reactive cysteine residues in proximity to the Ibrutinib binding site of proteins with which it interacts. All remaining reactive cysteine residues were then labeled with the novel 6-plex labeling reagents (FIG. 2B). Samples were mixed, inaccessible (disulfide bound) cysteine residues were reduced and alkylated with DTT/Iodoacetamide, and proteins were digested with trypsin. The resulting tryptic peptides were incubated with streptavidin agarose for enrichment of cysteine-containing peptides. Samples were analyzed by LC-MS, and the relative abundance of each cysteine-containing peptide across the mixed samples was quantified using the isobaric region reporter ions. Protein targets of Ibrutinib were identified by a reduction in signal in the samples treated with the drug, corresponding to drug engagement at the cysteine of interest, and concomitant reduction in the amount of signal for the desthiobiotin labeled peptide (FIG. 2E (upper panel), #—trifunctional compound disclosed herein). FIG. 2E, a cysteine-containing peptide from a known target of Ibrutinib displayed the expected reduction in signal in response to drug treatment. The use of the trifunctional compound disclosed herein resulted in significantly more dataset-wide agreement between DMSO and drug treatment, suggesting better discriminatory ability between true targets of the drug and experimental artifacts.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention. The indefinite articles “a” and “an.” as used herein in the specification and in the claims, unless clearly indicated to the contrary, should be understood to mean “at least one.” The phrase “and/or.” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B.” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of.” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other clements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of using a collection of compounds, comprising:
combining a first compound of the collection of compounds with a first sample comprising a first protein and/or peptide in a first container, and combining a second compound of the collection of compounds with a second sample comprising a second protein and/or peptide in a second container;
wherein each compound in the collection of compounds comprises moiety X (X), moiety Y (Y), and moiety Z (Z);
wherein moiety X comprises an isobaric region, moiety Y comprises an enrichment handle, and moiety Z comprises a thiol-reactive group;
wherein moiety Y is the same in each compound in the collection;
wherein moiety Z is the same in each compound in the collection; and
wherein moiety X differs only in a different placement of heavy isotopes for at least two compounds in the collection.

2. A collection of compounds, wherein each compound comprises:
moiety X (X);
moiety Y (Y); and
moiety Z (Z);
wherein moiety X comprises an isobaric region, moiety Y comprises an enrichment handle; and moiety Z comprises a thiol-reactive group;
wherein moiety Y is the same in each compound in the collection,
wherein moiety Z is the same in each compound in the collection, and
wherein moiety X differs only in a different placement of heavy isotopes for at least two compounds in the collection.

3. The collection of compounds of claim 2, wherein each compound further comprises one or more linkers.

4. The collection of compounds of claim 3, wherein the one or more linkers comprises an aliphatic chain.

5. The collection of compounds of claim 4, wherein the aliphatic chain comprises an alkane or heteroalkane chain.

6. The collection of compounds of claim 5, wherein the aliphatic chain comprises the heteroalkane chain, and the heteroalkane chain comprises a polyethylene glycol chain.

7. The collection of compounds of claim 2, wherein Y comprises a hexapeptide of histidine residues, a phosphorylated region, a fluoroalkane, 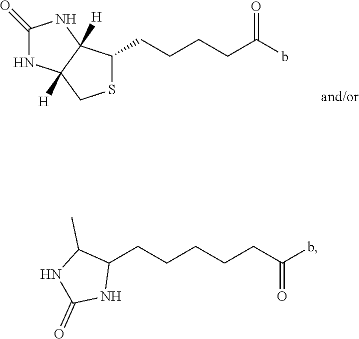
where “b” indicates the point of attachment to the remainder of the compound.

8. The collection of compounds of claim 2, wherein Y comprises 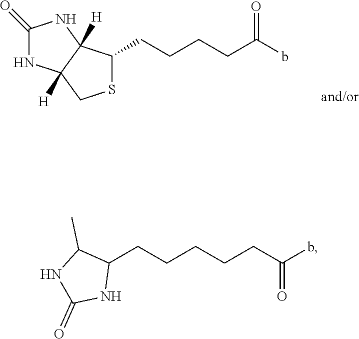
where “b” indicates the point of attachment to the remainder of the compound.

9. The collection of compounds of claim 2, wherein Z comprises iodoacetamide, maleimide, chloroacetamide, and/or acrylamide.

10. The collection of compounds of claim 2, wherein the compounds are of the formula:

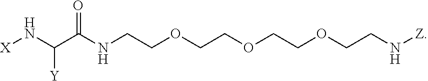

11. The collection of compounds of claim 2, wherein the compounds are of the formula: 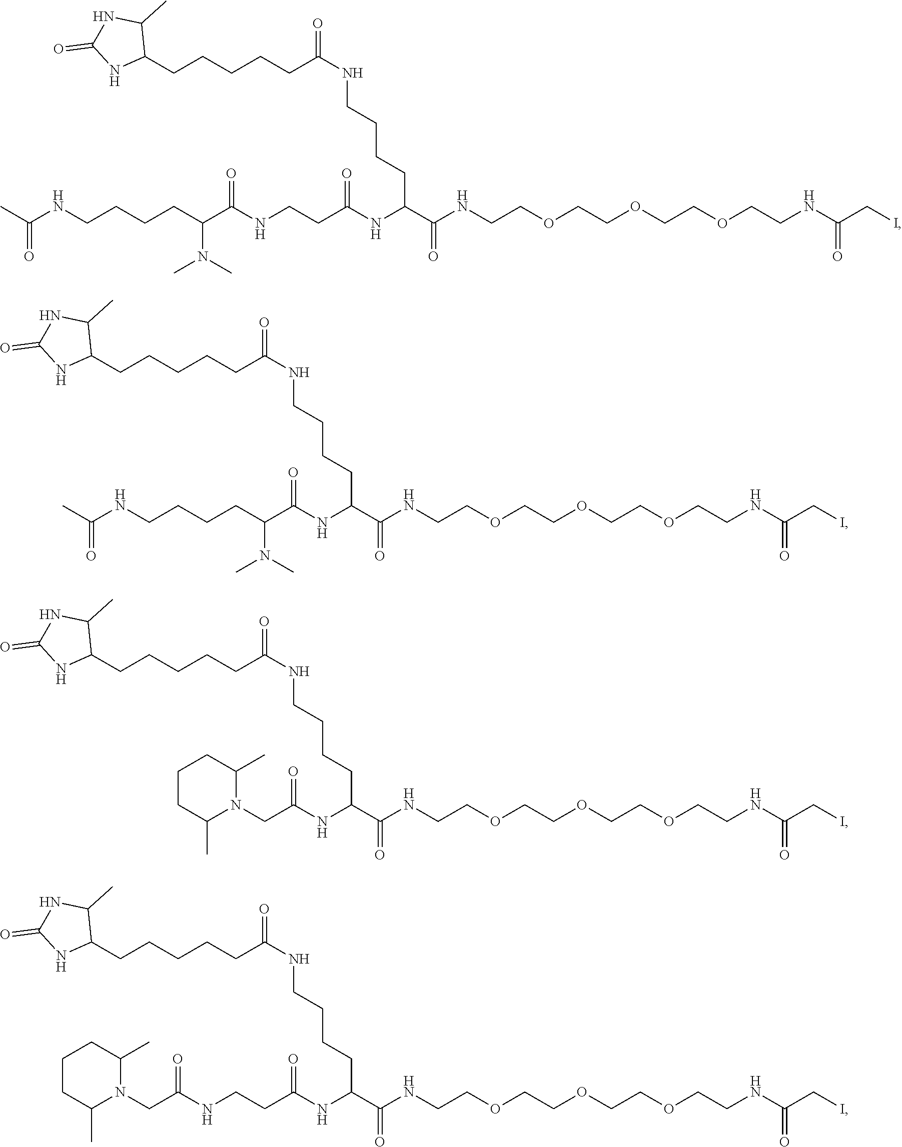 [SMILES: CC(=O)NCCCCC(C(=O)NCCC(=O)NC(CCCCNC(=O)CCCCCC1NC(=O)NC1C)C(=O)NCCOCCOCCOCCNC(=O)CI)N(C)C ; CC(=O)NCCCCC(C(=O)NC(CCCCNC(=O)CCCCCC1NC(=O)NC1C)C(=O)NCCOCCOCCOCCNC(=O)CI)N(C)C ; CC1NC(=O)NC1CCCCCC(=O)NCCCCC(NC(=O)CN1C(C)CCCC1C)C(=O)NCCOCCOCCOCCNC(=O)CI ; CC1NC(=O)NC1CCCCCC(=O)NCCCCC(NC(=O)CCNC(=O)CN1C(C)CCCC1C)C(=O)NCCOCCOCCOCCNC(=O)CI] 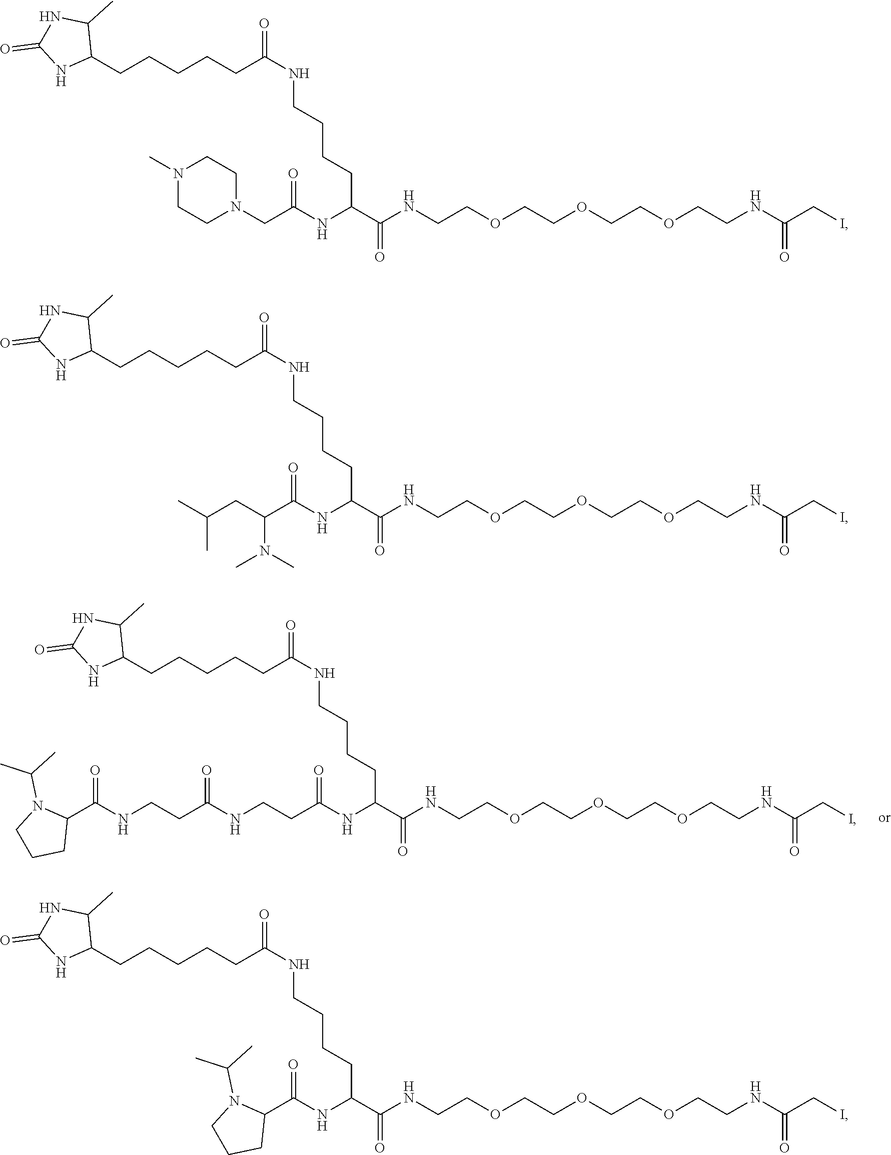 [SMILES: CC1NC(=O)NC1CCCCCC(=O)NCCCCC(NC(=O)CN1CCN(C)CC1)C(=O)NCCOCCOCCOCCNC(=O)CI ; CC(C)CC(C(=O)NC(CCCCNC(=O)CCCCCC1NC(=O)NC1C)C(=O)NCCOCCOCCOCCNC(=O)CI)N(C)C ; CC1NC(=O)NC1CCCCCC(=O)NCCCCC(NC(=O)CCNC(=O)CCNC(=O)C1CCCN1C(C)C)C(=O)NCCOCCOCCOCCNC(=O)CI ; "or" ; CC1NC(=O)NC1CCCCCC(=O)NCCCCC(NC(=O)C1CCCN1C(C)C)C(=O)NCCOCCOCCOCCNC(=O)CI]
or isotopically enriched derivatives thereof.

12. A method of using the collection of compounds of claim 2, wherein the method comprises combining a first compound of the collection of compounds with a first sample comprising a first protein and/or peptide in a first container, and combining a second compound of the collection of compounds with a second sample comprising a second protein and/or peptide in a second container.

13. The method of claim 1, wherein the first compound of the collection of compounds covalently attaches to one or more cysteine residues of the first protein and/or peptide and/or the second compound of the collection of compounds covalently attaches to one or more cysteine residues of the second protein and/or peptide.

14. The method of claim 1, further comprising combining the contents of the first container and the second container into a third container.

15. The method of claim 1, wherein the method further comprises reducing and/or alkylating at least one cysteine residue on the proteins and/or peptides.

16. The method of claim 1, wherein the method further comprises digesting at least one of the proteins and/or peptides.

17. The method of claim 15, wherein the reduction, alkylation, and digestion steps occur after the combination of the samples with the compounds of the collection.

18. The method of claim 15, wherein the reduction, alkylation, and digestion steps occur after the combining of the contents of the first container and the second container into the third container.

19. The method of claim 14, further comprising contacting the contents of the third container with a substrate, such that moiety Y preferentially binds to the substrate.

20. The method of claim 19, wherein the substrate comprises avidin, streptavidin, a chelated metal, and/or a fluorous resin.

21. The method of claim 19, wherein the contacting the contents of the third container with the substrate occurs after the reduction, alkylation, digestion, and combining the samples with the compounds of the collection steps.

22. The method of claim 1, further comprising analyzing the samples with mass spectrometry.

23. The method of claim 22, wherein the analyzing the samples with mass spectrometry occurs after the reduction, alkylation, digestion, contacting the contents of the containers with the immobilized substrate, and combining the samples with the compounds of the collection steps.

24. The method of claim 1, wherein the method comprises analyzing multiple samples simultaneously.

25. The method of claim 24, wherein the method comprises analyzing greater than or equal to 5 samples simultaneously.

26. The method of claim 24, wherein the method comprises analyzing less than or equal to 100 samples simultaneously.

27. The method of claim 1, wherein the method further comprises determining whether a target is bound to one or more cysteine residues on the one or more proteins and/or peptides.

28. The method of claim , wherein the target is a pharmaceutical drug candidate and/or wherein the method comprises screening a library of pharmaceutical drug candidates.

29. A system comprising the collection of compounds of claim 2 and a substrate to which moiety Y selectively binds.

30. A kit comprising the collection of compounds of claim 2 and instructions for use.

31. The kit of claim 30, further comprising one or more buffers.

32. The kit of claim 31, further comprising one or more microcentrifuge tubes.

33. The kit of claim 32, further comprising one or more spin filters.

34. The kit of claim 33, further comprising a control sample.

35. The kit of claim 34, further comprising a substrate to which moiety Y (Y) selectively binds.

36. A compound, comprising:
moiety X (X);
moiety Y (Y); and
moiety Z (Z);
wherein moiety X comprises an isobaric region, moiety Y comprises an enrichment handle; and moiety Z comprises a thiol-reactive group.
